(19) United States
(12) Patent Application Publication
(10) Pub. No.: US 2008/0242836 A1
(43) Pub. Date: Oct. 2, 2008

(54) Convergent Solid Phase Peptide Synthesis By Reaction Of Two Fragments Bound To Solid Support

(75) Inventor: Ram Prakash Sharma, Southampton (GB)

Correspondence Address: STEPTOE & JOHNSON LLP, 1330 CONNECTICUT AVENUE, N.W., WASHINGTON, DC, 20036, US

(73) Assignee: UNIVERSITY OF SOUTHAMPTON, SOUTHAMPTON (GB)

(21) Appl. No.: 11/886,180

(22) PCT Filed: Mar. 13, 2006
(86) PCT No.: PCT/GB2006/000862

§ 371 Date: Jun. 2, 2008

(30) Foreign Application Priority Data
Mar. 14, 2005 (GB) 0505200.6

(51) Int. Cl. C07K 14/00; C07K 1/00; C07K 7/06

(52) U.S. Cl. 530/325; 530/334; 530/328

(57) ABSTRACT

A process for preparing a peptide or protein by solid phase synthesis comprising combining a sequence including one or more amino acids obtainable by C-N synthesis linked to a first resin, with an amino acid sequence including one or more amino acid obtainable by N-C synthesis linked to a second resin so as to create a native peptide link between unprotected N and unprotected C terminals of said amino acid sequences, and optionally releasing the resulting peptide from one or more of the linked resins so as to combine with further N-C or C-N sequences or to release the desired peptide or protein sequence.

The present invention relates to a process for the preparation of peptides and proteins, to peptides and proteins obtained by such processes, and to intermediates useful in such processes.

Peptides and proteins are composed of amino acids. There are about 20 different amino acids commonly occurring in nature, and they are linked together in chains to form peptides. Biologically active peptides, consisting of between 2 and 50 amino acids, span a wide range of functions in nature: hormones, chemokines, neurotransmitters, cytokines and immunological agents being among them. They have also been shown to be effective as prophylactic and therapeutic vaccines as well as enzyme inhibitors.

Protein therapeutics has emerged as one of the most promising segments of the pharmaceutical market since the introduction of recombinant insulin in 1982. To produce these important drugs commercially, companies have focused to date on biological approaches such as recombinant-DNA expression methods (microbial fermentation and mammalian cell culture) and native protein isolation. However numerous problems are associated with these methods:

a) limited supply of product is possible
b) viral contamination risk
c) product heterogeneity
d) inability to produce some proteins e.g. those that are toxic to the cell
e) non-human post-translational modifications, i.e. incorrect glycosylation or folding
f) time-consuming
g) structural modifications are limited to the 20 naturally occurring amino acids.

Chemical protein synthesis provides a rapid and efficient route for the production of homogenous proteins containing up to 250 amino acids that are free of biological contaminants. In this field the development of solid-phase peptide synthesis (SPPS) by Merrifield in 1963 merited the award of Nobel prize in 1984 [Merrifield, R. B. (1963) J. Amer. Chem. Soc., 85, 2149-2154.]. This method is still widely used. However this method originally only allowed efficient production of small peptides, for example up to about 10 kDa, such as hormones and cytokines. Another significant limitation of this method is incomplete synthesis and side reactions.

The present inventors have made advances towards reversing the conventional C-to-N direction of synthesis and a new approach to synthesising peptides to allow the preparation on the solid-phase of peptide analogues possessing C-terminal modifications (such as esters, thioesters, alcohols, aldehydes and others), peptides possessing peptide bond modifications (such as reduced peptide bonds, urea, and isosteres) as well as to facilitate fragment coupling on the solid-phase. This N to C method was first described in Sharma, R. P., Jones, D. A., Corina, D. L. and Akhtar, M. (1994) in Peptides: Chemistry, Structure and Biology, Proceedings of the Thirteenth American Peptide Symposium (Hodges, R. S, and Smith, J. A., eds.), pp. 127-129, ESCOM, Leiden; Jones, D. A. (1993) PhD Thesis, University of Southampton; and WO93/65065.

In the past, the lack of suitable protection for the carboxyl group which could be removed under mild conditions had hindered progress in this area. Earlier attempts at the solid-phase peptide synthesis in the N-to-C direction are described in Letsinger, R. L. and Kornet, M. J. (1963) J. Amer. Chem. Soc., 85, 3045-3046; Letsinger, R. L., Kornet, M. J., Mahadevan, V. and Jerina, D. M. (1964) J. Amer. Chem. Soc., 86, 5163; and Felix, A. M. and Merrifield, R. B. (1970) J. Amer. Chem. Soc., 92, 1385-1391. These methods were hindered by the use of amino acid esters that were effectively too stable. The conditions required for the removal of the ester protection before commencing the next addition cycle as a consequence were very harsh. In order to improve this situation the present inventors employ more suitable amino acid ester building blocks and have developed the use of trialkoxy silyl (tBos) esters of amino acids for use in solid-phase peptide synthesis in the N-to-C direction. These derivatives can be readily prepared, are inexpensive, stable throughout the coupling reaction and the protecting group can be selectively removed in high yield under mild acid conditions before commencing the next cycle.

Another known method of peptide or protein synthesis is known as native chemical ligation. This process uses thioester linked intermediates which undergo spontaneous rearrangement and form native peptide bonds at the ligation site [see US 2002/0132975; Canne et al (1999) Chemical Protein Synthesis by Solid Phase Ligation of Unprotected Peptide Segments, J. Am. Chem. Soc. 121, 8720-8727 and Kawakami, T. and Aimoto, S., (2003), Tetrahedron Letters, 44:6059-6061].

The Canne et al method provides solid phase sequential chemical ligation of peptide segments in a N-terminus to C-terminus direction, with the first solid phase bound unprotected segment bearing a C-terminal a thioester that reacts with another unprotected peptide segment containing an N-terminal cysteine.

The present invention provides novel techniques for the synthesis of peptides and proteins without the limitations and disadvantages of previous methods.

In a first aspect, the present invention provides a process for the preparation of a solid support-bound peptide of formula (I)

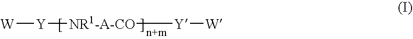

wherein

n is a positive integer

m is a positive integer

W and W′ are solid supports

Y and Y′ are linker groups

R¹ is hydrogen or a substituent, and may be the same or different,

and for each A, which may be the same or different,
i) A represents the amino acid residue; or
ii) A, taken together with R¹ and N, forms a heterocycle (for instance in the case of proline);
   comprising reacting a solid support-bound activated peptide of formula (II) with a solid support-bound peptide of formula (III);

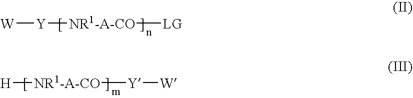

wherein LG is a leaving group.

By use of the term “solid support” we mean the support onto which the amino acids are linked, optionally through a linker. The supports include solid and soluble solid materials or matrixes, and resins. Preferably, the solid support is insoluble in the solvents in which the desired reactions take place.

Preferred solid supports W for N-C synthesis are derivatised Merrifield resins, that is resins based on chloromethylstyrene/divinylbenzene copolymers. Particularly useful resins are PEG-PS e.g. Tentagel (obtained from Novabiochem) which have increased tolerance to aqueous media.

By “leaving group” it is meant any chemical moiety which is capable of detachment from the acyl group of the amino acid with the concomitant formation of a new amide bond. Many suitable leaving groups will be known to those skilled in the art. Examples of particularly suitable leaving groups include:

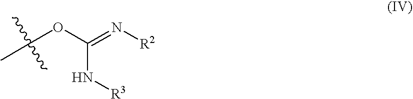

i) derivatives of carbodiimides of formula (IV)
wherein R² and R³ are independently C₁₋₁₀ hydrocarbyl groups, preferably cyclohexyl;
ii) derivatives of pentafluorophenol, hydroxybenzatriazole, hydroxysuccinimide, 1-hydroxy-7-azabenzotriazole, carbonyldiimidazole, 3-hydroxy-3,4-dihydro-4-oxo-1,2,3-benzotriazine, N-ethyl-5-phenylisoxazolium-3′-sulphonate;
iii) halides, particularly fluoride.

A particularly preferred leaving group is oxybenzotriazole (—OBt).

Preferably, the solid support-bound activated peptide of formula (II) is prepared by treatment of the corresponding solid support-bound peptide of formula (V) with an activating agent.

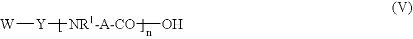

By “activating agent” it is meant any reagent or combination of reagents that is capable of converting the free carboxylic acid group of an amino acid or peptide fragment to an activated form, in which the acyl carbon bears a leaving group LG as defined above. Many activating agents have proved useful in this capacity, and the skilled man will have little difficulty in selecting an appropriate one.

Preferred activating agents are selected from:

i) carbodiimides, including 1,3-dicyclohexylcarbodiimide (DCC); 1-ethyl-3-(3′-dimethylaminopropyl)carbodiimide hydrochloride, (EDCI), optionally with base;
ii) aminium/uronium based reagents, including 1-benzotriazol-1-yloxy-bis(pyrrolidino)uronium hexafluorophosphate, 5-(1H-benzotriazol-1-yloxy)-3,4-dihydro-1-methyl 2H-pyrrolium hexachloroanitimonate, benzotriazol-1-yloxy-N,N-dimethylmethaniminium hexachloroantimonate, O-(7-azabenzotriazol-1-yl)-1,1,3,3-tetramethyluronium hexafluorophosphate, O-(7-azabenzotriazol-1-yl)-1,1,3,3-bis(tetramethylene)uronium hexafluorophosphate, O-(benzotriazol-1-yl)-1,1,3,3-tetramethyluronium hexafluorophosphate, O-(7-azabenzotriazol-1-yl)-1,1,3,3-bis(pentamethylene)uronium tetrafluoroborate, 2-(3,4-dihydro-4-oxo-1,2,3-benzotriazin-3-yl)-1,1,3,3-tetramethyluronium tetrafluoroborate, 2-(5-norbornene-2,3-dicarboximido)-1,1,3,3-tetramethyluronium tetrafluoroborate, 2-(2-oxo-1(2H)-pyridyl-1,1,3,3-tetramethyluronium tetrafluoroborate, 2-succinimido-1,1,3,3-tetramethyluronium tetrafluoroborate, optionally in combination with base;
iii) phosphonium based reagents including O-(7-azabenzotriazol-1-yl)-tris(dimethylamino)phosphonium hexafluorophosphate, benzotriazol-1-yl diethyl phosphate 1-benzotriazolyoxytris(dimethylamino)phosphonium hexafluorophosphate (Castro's Reagent), 7-azobenzotriazolyoxytris(pyrrolidino)phosphonium hexafluorophosphate, 1-benzotriazolyoxytris(pyrrolidino)phosphonium hexafluorophosphate, optionally in combination with base;
iv) other peptide coupling reagents including 2-bromo-3-ethyl-4-methyl thiazolium tetrafluoroborate, bis(2-oxo-3-oxazolidinyl)phosphinic chloride, bromotris(dimethylamino)phosphonium hexafluorophosphate, bis(tetramethylenefluoroformamidinium) hexafluorophosphate, 2-chloro-1,3-dimethylimidazolidinium hexafluorophosphate, 3-(diethoxyphosphoryloxy)-1,2,3-benzotriazin-4(3H)-one, diphenylphosphinic chloride, 2-ethoxy-1-ethoxycarbonyl-1,2-dihydroquinoline, pentafluorophenyl diphenylphosphinate, S-(1-oxido-2-pyridinyl)-1,1,3,3-tetramethylthiouronium hexafluorophosphate, bromotris(pyrrolydino)phosphonium hexafluorophosphate, chlorotris(pyrrolydino)phosphonium hexafluorophosphate, tetramethylfluoroformamidinium hexafluorophosphate, S-(1-oxido-2-pyridinyl)-1,1,3,3-tetramethylthiouronium tetrafluoroborate, optionally in combination with base.

Optionally, the activating agent includes at least one activating additive. Preferred activating additives include pentafluorophenol, hydroxybenzatriazole, hydroxysuccinimide, 1-hydroxy-7-azabenzotriazole, carbonyldiimidazole, 3-hydroxy-3,4-dihydro-4-oxo-1,2,3-benzotriazine or N-ethyl-5-phenylisoxazolium-3′-sulphonate.

A preferred activating agent is a combination of 1-benzotriazolyoxytris(dimethylamino)phosphonium hexafluorophosphate (BOP, Castro's Reagent) and diisopropylethylamine.

The skilled person will readily appreciate that the activation of (V) above to give (II) above, and the coupling of (II) with (III) can be carried out in one operational step; for instance, the solid support-bound (V) may be activated in the presence of (III), without the necessity of isolating activated form (II).

Preferably, the reaction of (II) with (III) to give (I) occurs in DMF.

The skilled person will moreover appreciate that before and after each step it may be necessary to swell the resin with a suitable solvent to enable reagent(s) to permeate fully and react completely with the bound peptide. Furthermore, after each step it may be necessary or expedient to wash the resin to remove excess reagent, byproducts and impurities.

In a second aspect, the invention relates to a solid support bound peptide of formula (I) as defined above.

In a third aspect, the invention relates to process for the preparation of a compound of formula (VI)

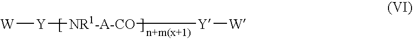

wherein W, Y, W′, Y′, R¹, A, n and m are as defined above, and x is a positive integer;
comprising the steps of
(a) reacting a solid support-bound activated peptide of formula (II) with a solid support-bound peptide of formula (III);

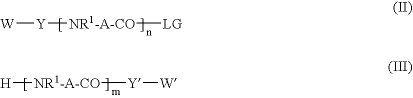

to give a solid support-bound peptide of formula (I)

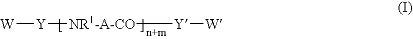

(b) cleaving the peptide (I) from the support W′ (and linker Y′) to give a solid support-bound peptide of formula (VII)

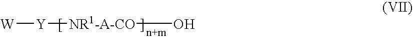

(c) treating solid support-bound peptide of formula (VII) with an activating agent to give a solid support-bound activated peptide of formula (VIII)

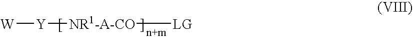

wherein LG is as defined above;
(d) repeating steps (a), (b) and (c) x times.

In a fourth aspect, the invention relates to process for the preparation of a compound of formula (VI)

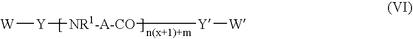

wherein W, Y, W′, Y′, R¹, A, n and m are as defined above and x is a positive integer; comprising the steps of
(a) reacting a solid support-bound activated peptide of formula (II) with a solid support-bound peptide of formula (III);

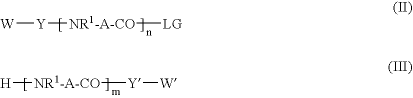

to give a solid support-bound peptide of formula (I)

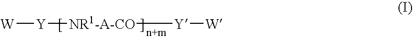

(b) cleaving the peptide (I) from the support W (and linker Y) to give a solid support-bound peptide of formula (IX)

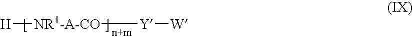

(c) repeating steps (a) and (b) x times.

In a fifth aspect, the present invention provides a process for preparing a peptide or protein by solid phase synthesis comprising combining a sequence including one or more amino acids obtainable by C-N synthesis linked to a first resin, with an amino acid sequence including one or more amino acids obtainable by N-C synthesis linked to a second resin so as to create a peptide link between unprotected N and unprotected C terminals of said amino acid sequences, and optionally releasing the resulting peptide from one or more of the linked resins so as to combine with further N-C or C-N sequences or to release the desired peptide or protein sequence.

The amino acids can be natural, unnatural or modified. The residues A of the amino acids may incorporate protected functional groups. Preferably, the amino acids are a amino acids, although β and other amino acids may also be employed.

In a preferred aspect the completed peptide is cleaved from the solid supports W and W′ to give a peptide of formula (X) or a salt form thereof.

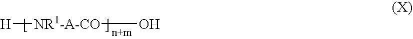

In a preferred aspect, the completed peptide is released from the solid support at only the C-terminus, to give an N-terminal resin bound peptide (VII) or a salt form thereof.

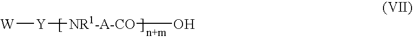

In a preferred aspect, the completed peptide is released from the solid support at only the N-terminus, to give an C-terminal resin bound peptide (IX) or a salt form thereof.

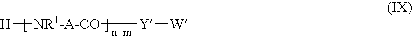

A particularly surprising feature of the invention is that two amino acid sequences have been found to ligate efficiently to produce a native peptide link irrespective of the amino acids involved without the need to cleave the peptides from their resins. Without wishing to be limited by any such theory, this would appear to be through a surface reaction mechanism, which hitherto would be counter to expectation.

Preferred solid supports W are derivatised Merrifield resins, that is resins based on chloromethylstyrene/divinylbenzene copolymers. Particularly useful resins are PEG-PS e.g. Tentagel (obtained from Novabiochem) which have increased tolerance to aqueous media. A particularly preferred resin is MBHA (4-methylbenzhydrylamine).

The linker group Y is a chemical bond, or chemical moiety capable of forming a covalent bond to both the solid support W and the amine group of an amino acid. Many suitable linker groups are known. Preferably, the Y—N bond of compound (I) above is cleavable to yield a solid support-bound peptide of formula (IX).

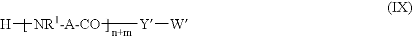

A preferred linker group Y is formylmethyl —CH₂—O—(C═O)—.

Preferred solid supports W′ are derivatised Merrifield resins, that is resins based on chloromethylstyrene/divinylbenzene copolymers.

The linker group Y′ is a chemical bond, or chemical moiety capable of forming a covalent bond to both the solid support W′ and the carboxylic acid group of an amino acid. Many suitable linker groups are known. Preferably, the Y′—C bond of compound (I) above is cleavable to yield a solid support-bound peptide of formula (VII).

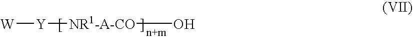

In a preferred embodiment, the linker groups Y and Y′ are selected such that the bond N—Y can be cleaved under conditions to which the C—Y′ bond is stable. In this context, “stable” means that the C—Y′ bond undergoes less than 20% cleavage; preferably less than 10% and most preferably less than 5%.

In a preferred embodiment, the linker groups Y and Y′ are selected such that the bond C—Y′ can be cleaved under conditions to which the bond N—Y is stable. In this context, “stable” means that the N—Y bond undergoes less than 20% cleavage; preferably less than 10% and most preferably less than 5%.

Conditions for the cleavage of the Y—N and C—Y′ bond will depend on the nature of the groups Y and Y′.

Suitable linker groups Y′ include (a), (b), (c), (d) and (e) and FMOC derived linkers.

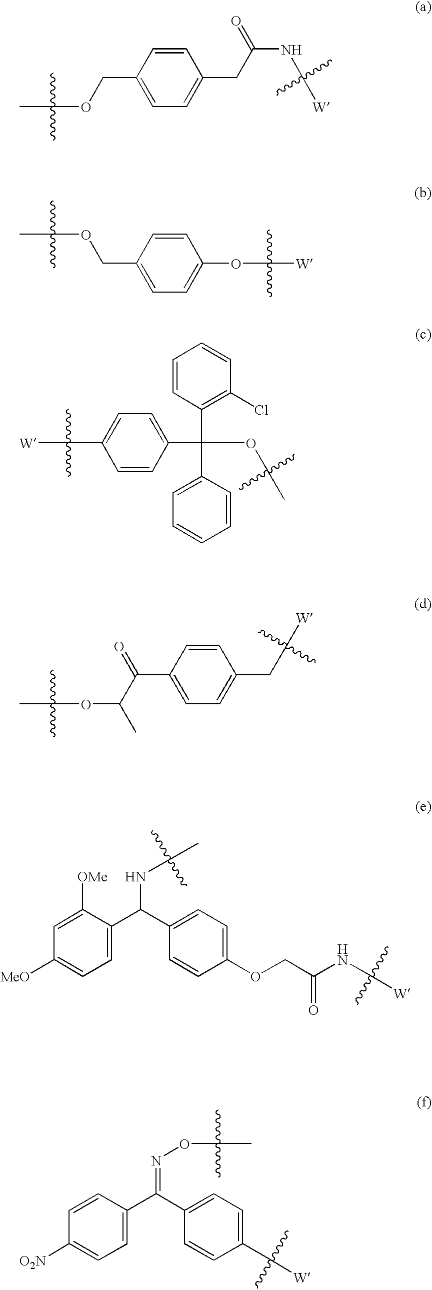

Preferably, the linker Y′ is (b).

As will be appreciated, certain solid supports W and W′ are commercially available derivatised with linker groups Y and Y′. For example, chloromethylstyrene/divinylbenzene copolymers attached to linker (a) are known as PAM resins; those attached to (b) as WANG resins; those attached to (c) as trityl resins; those attached to (e) as RINK resins; and those attached to (f) as oxime resins.

In a preferred embodiment, the free peptide (X) may be cleaved from both solid supports W and W′ (and linkers Y and Y′) in one step from the solid support-bound peptide (I). Preferred conditions for achieving this are treatment with HF, HBr or trifluoromethanesulfonic acid (TFSA). Particularly preferably, this cleavage occurs with simultaneous deprotection of one, more than one or all of the protected side chains A of the amino acids (where present).

Many suitable methodologies for the synthesis of solid support-bound peptides (III) are described in the art, and in particular in Barany, G. and Merrifield, R. B. (1979) in The Peptides (Groaa, E. and Meienhofer, J. eds.), vol 2, pp. 1-284, Acadmic Press, New York, and Atherton, E. and Sheppard, R. C. (1989) in Solid Phase Peptide Synthesis: A Practical Approach, IRL Press, Oxford. Particularly preferred methods are described in the examples herein.

Suitable methods for assembling solid support-bound peptides (V) are those described in Sharma, R. P., Jones, D. A., Corina, D. L. and Akhtar, M. (1994) in Peptides: Chemistry, Structure and Biology, Proceedings of the Thirteenth American Peptide Symposium (Hodges, R. S, and Smith, J. A., eds.), pp. 127-129, ESCOM, Leiden; Jones, D. A. (1993) PhD Thesis, University of Southampton; and WO93/65065. Another recent method is described in Canne & Kent 1999 as mentioned above.

Preferably, R¹ is hydrogen, hydrocarbyl, or A, taken together with R¹ and N, forms a heterocycle. More preferably, R¹ is hydrogen, C₁₋₆ alkyl, or C₁₋₆ acyl, or A, taken together with R¹ and N, forms a heterocycle. A preferred heterocycle is pyrrolidine.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group. Preferred hydrocarbyl groups are those comprising 1 to 10 carbon atoms.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

The present invention will be described in more detail with reference to the following non-limiting examples.

### EXAMPLES

Abbreviations: DCC, N,N′dicyclohexylcarbodiimide; SPPS, solid-phase peptide synthesis; RP-HPLC, reversed-phase high-performance liquid chromatography; HOBt, 1-hydroxybenzotriazole; BOP, benzotriazole-1-yloxytris(dimethylamino)phosphonium hexafluorophosphate; TLC, thin-layer chromatography; FIB-MS, fast ion bombardment mass spectrometry; ES-MS, electrospray mass spectrometry; MALDI-TOF, matrix assisted laser desorption ionisation-time of flight; DIPEA, diisopropylethylamine; DMAP, N,N′-dimethylaminopyridine; Boc, tert-butyloxycarbonyl; Fmoc, 9-fluorenylmethoxycarbonyl; DCM, dichloromethane; TFMSA, trifluoromethane sulfonic acid; DMF, N,N-dimethylformamide; HVTLE, high voltage thin-layer electrophoresis; Tbos, tri-tert-butoxysilyl; TFA, trifluoroacetic acid; HF, hydrogen fluoride; PEG2000-OH, Polyethylene glycol 2000 monomethyl ether; HCTU, O-(1H-6-Chlorobenzotriazole-1-yl)-1,1,3,3-tetramethyluronium hexafluorphosphate; other abbreviations correspond to standard nomenclature used for naturally occurring amino acids. All amino acids except glycine are of the L-configuration unless otherwise specified. [Standard abbreviations for amino acids, peptides and protecting groups follow the recommendations of the IUPAC-IUB Joint Commission on Biochemical Nomenclature (1984) Eur. J. Biochem. 138, 9-37.]

### General Materials and Methods

Unless otherwise stated, all solvents and reagents obtained from various commercial sources were of highest grade and were used without further purification. DMF was stored over molecular sieve 4 Å. Proton nuclear magnetic resonance spectra were recorded on a Hitachi Perkin Elmer R1500 60 MHz instrument in CDCl₃ and chemical shifts are reported as 6 units (ppm) relative to tetramethyl silane. TLC was performed on Merck 60 F₂₅₄ precoated silica gel plates using solvent systems (v/v): (A) CHCl₃-MeOH, 9:1 and (B) diethylether-light petroleum (60-80° C.), 7:3. Reaction products were visualised by UV fluorescence (254 nm), 2% ninhydrin in ethanol or by using iodine vapour. Column chromatography was carried out on Merck (230-400 mesh) silica gel. Optical rotations were measured on a Perkin Elmer 141 polarimeter (sodium lamp, 589 nm) at 21° C. Analytical and preparative reversed-phase HPLC (RP-HPLC) experiments were performed on a Gilson 715 instrument equipped with a multi-wave length detector (Applied Biosystems 759A) and two slave 306 pumps. Retention times are given for gradient elution using the following conditions: Column, Vydac C₁₈ (10 m, 0.46 and 2.2×25 cm); eluant A, 0.1% (v/v) TFA in H₂O; eluant B, 0.1% (v/v) TFA in acetonitrile; gradient, 0% over 2 min., 0-80% over 32 min., flow rate, 1 ml/min (analytical) and 10 ml/min (preparative); absorbance, 216 and 235 nm. Molecular weight determinations were carried out by fast ion bombardment (FIB), on a TS250 VG, matrix assisted laser desorption ionisation-time of flight (MALDI-TOF), Perceptives Biosystems Voyager and electrospray (ES) Micromass Quattro 11 mass spectrometers. Infrared spectra were recorded as thin film or in Nujol mull on a Pye Unicam SP3-200 instrument. The accurate mass determination of TBos amino acid esters were performed using a direct probe (EI) approach with suitable internal standards.

### Preparation of Amino Acid Tri-Tert-Butoxysilyl Esters (TBos Esters): General Procedure

An amino acid (50 mmol) was suspended in tert-butanol (40 ml) containing anhydrous pyridine (16.8 ml, 210 mmol) in a 250 ml two-necked flask fitted with a calcium chloride drying tube and a rubber septum. The mixture was cooled to 0° C. (ice bath) and stirred. Silicon tetrachloride (5.73 ml, 50 mmol) was added drop wise with care and was stirred for two hours at room temperature and then at 50° C. for a further 30 minutes. The mixture was allowed to cool; the precipitated pyridinium hydrochloride filtered through a bed of Celite and was washed with ethyl acetate (50 ml). The solvents were removed under reduced pressure (rotary evaporation) and the resultant oily residue dissolved in ethyl acetate (50 ml), transferred to a separating funnel and was washed with H₃PO₄ solution (1M, 10 ml), water (10 ml), NaHCO₃ (10% w/v, 20 ml), brine (2×10 ml), and then dried (Na₂SO₄). Removal of the solvents under reduced pressure gave the target compound as a syrup which was used without further purification for peptide synthesis or stored under argon at 4° C. until further use. On prolonged storage, TBos esters have been found to give a white precipitate of a polymeric form of silicon oxide, which can easily be removed by dissolving the ester in acetonitrile followed, by filtration and removal of solvent under reduced pressure. All TBos esters were purified by flash column chromatography using silica gel, which was pre-washed with 1% triethylamine in chloroform. The required ester was obtained by eluting with chloroform-methanol (95:5, v/v). The TBos esters of all naturally occurring amino acids were synthesised in this manner and gave satisfactory HPLC and TLC analysis.

Alanine-tri-tert-butoxysilyl ester. Oil; Yield 11.0 g, 66%; Rf_{A}=0.69; ESMS, m/z 336 [M+H]⁺; IR (thin film) 3450-3320 (br, NH₂), 1750 (s, C═O str, ester), 1260, 1195 (s, C—O), 1110 (s, Si—O) cm⁻¹; ¹H-NMR (60 MHz, CDCl₃ [symbol] 1.14 (s, 27H, Si[OC(CH₃)₃]₃), 1.6 to 1.8 (d, 3H, CH₃) and 3.85 to 4.15 (m, 1H, α-proton).

Arginine-(N^{G}—NO₂)-tri-tert-butoxysilyl ester. Oil; Yield 12.4 g, 53%; Rf_{A}=0.34; ESMS, m/z 466 [M+H]⁺; IR (thin film) 3500-3200 (br, NH₂), 1750 (s, C═O str, ester), 1260, 1100 (s, C—O), 970 (s, Si—O) cm⁻¹.

Asparagine-tri-tert-butoxysilyl ester. Oil; Yield 14.0 g, 74%; Rf_{A}=0.59; ESMS, m/z 379 [M+H]⁺; IR (thin film) 3500-3300 (br, NH₂), 1750 (s, C═O str, ester), 1265, 1100 (s, C—O), 970 (s, Si-0) cm⁻¹.

Aspartate-(OBzl)-tri-tert-butoxysilyl ester. Oil; Yield 19.7 g, 83%; Rf_{A}=0.71; ESMS, m/z 470 [M+H]⁺; IR (thin film) 3450-3320 (br, NH₂), 1750 (s, C═O str, ester), 1260, 1195 (s, C—O), 1110 (s, Si-0) cm⁻¹.

Cysteine-(MeOBzl)-tri-tert-butoxysilyl ester. Oil; Yield 19.8 g, 81%; Rf_{A}=0.68; ESMS, m/z 488 [M+H]⁺; IR (thin film) 3450-3300 (br, NH₂), 1740 (s, C═O str, ester), 1250, 1180 (s, C—O), 1100 (s, Si—O) cm⁻¹; ¹H-NMR (60 MHz, CDCl₃) [symbol] 1.29 (s, 27H, Si [OC(CH₃)₃]₃), 3.05 to 3.2 (m, 2H, CH₂S), 3.76 (s, 2H, benzylic-CH₂), 3.85 (s, 3H, OCH₃), 4.15 (m, 1H, α-proton), 6.60 to 6.85 (d, 2H, aromatics) and 7.2 to 7.4 (d, 2H, aromatics).

Glutamate-(OBzl)-tri-tert-butoxysilyl ester. Oil; Yield 19.6 g, 81%; Rf_{A}=0.84; ESMS, m/z 484 [M+H]⁺; IR (thin film) 3420-3200 (br, NH₂), 1750, 1730 (s, C═O str, esters), 1265, 1170 (s, C—O), 1110 (s, Si—O) cm⁻¹; ¹H-NMR (60 MHz, CDCl₃) [symbol] 1.27 (s, 27H, Si [OC(CH₃)₃]₃), 1.82 to 2.75 (m, 4H, CH₂CH₂), 3.80 to 4.5 (m, 3H, benzylic-CH₂, α-proton) and 7.2 to 7.5 (m, 5H, aromatics).

Glutamine-tri-tert-butoxysilyl ester. Oil; Yield 8.2 g, 42%; Rf_{A}=0.40; ESMS, m/z 393 [M+H]⁺; IR (thin film) 3500-3200 (br, NH₂), 1730 (s, C═O str, ester), 1220, 1190 (s, C—O), 1090 (s, Si—O) cm⁻¹.

Glycine-tri-tert-butoxysilyl ester. Oil; Yield 9.89 g, 61%; Rf_{A}=0.68; ESMS, m/z 322 [M+H]⁺; IR (thin film) 3500-3320 (br, NH₂), 1760 (s, C═O str, ester), 1250, 1195 (s, C—O), 1090 (s, Si-0) cm⁻¹; ¹H-NMR (60 MHz, CDCl₃) [symbol] 1.32 (s, 27H, Si[OC(CH₃)₃]₃) and 3.80 to 4.10 (m, 2H, α-protons).

Histidine-(N^{lm}-DNP)-tri-tert-butoxysilyl ester. This derivative was prepared as described in the general procedure except that anhydrous pyridine (20.8 ml, 260 mmol) was used and gave yellow oil. Yield 10.2 g, 36%; Rf_{A}=0.60; ESMS, m/z 568 [M+H]⁺; IR (thin film) 3450-3220 (br, NH₂), 1750 (s, C═O str, ester), 1600, 1530, 1350 (imidazole, DNP), 1260, 1190 (s, C—O), 1080 (s, Si—O) cm⁻¹.

Histidine-(N^{lm}-Tosyl)-tri-tert-butoxysilyl ester. Anhydrous pyridine (20.8 ml, 260 mmol) was used in this preparation and gave an oil. Yield 12.9 g, 48%; Rf_{A}=0.58; ESMS, m/z 556 [M+H]⁺; IR (thin film) 3500-3300 (br, NH₂), 1740 (s, C═O str, ester), 1600 (imidazole), 1260, 1200 (s, C—O), 1100 (s, Si—O) cm⁻¹.

Isoleucine-tri-tert-butoxysilyl ester. Oil; Yield 15.1 g, 80%; Rf_{A}=0.67; ESMS, m/z 378 [M+H]⁺; IR (thin film) 3450-3300 (br, NH₂), 1750 (s, C═O str, ester), 1250, 1190 (s, C—O), 1090 (s, Si—O) cm⁻¹.

Leucine-tri-tert-butoxysilyl ester. Oil; Yield 13.8 g, 73%; Rf_{A}=0.71; ESMS, m/z 378 [M+H]⁺; IR (thin film) 3350-3200, 3000, 1750, 1265, 1155 and 1100 cm⁻¹. ¹H-NMR (60 MHz, CDCl₃) δ 0.91-1.0, (d, 7H), 1.27 (s, 27H), 1.35-1.74 (m, 2H), and 4.0-4.40 (m, 1H).

Lysine-(Z)-tri-tert-butoxysilyl ester. Oil; Yield 23.0 g, 87%; Rf_{A}=0.67; ESMS, m/z 527 [M+H]⁺; IR (thin film) 3500 (br, NH₂), 1755, 1720 (s, C═O str, ester, carbamate), 1265 (s, C—O), 1100 (s, Si—O) cm⁻¹.

Lysine (Fmoc)-tri-tert-butoxysilyl ester. Oil; Yield 18.7 g, 61%; Rf_{A}=0.71; ESMS, m/z 615 [M+H]⁺; IR (thin film) 3380 (br, NH₂), 1755, 1710 (s, C═O str, ester, Fmoc), 1265 (s, C—O), 1100 (s, Si—O) cm⁻¹.

Methionine-tri-tert-butoxysilyl ester. Waxy solid; Yield 13.8 g, 70%; Rf_{A}=0.55; ESMS, m/z 395 [M+H]⁺; IR (thin film) 3500-3400 (br, NH₂), 1730 (s, C═O str, ester), 1230 (s, C—O), 1080 (s, Si—O) cm⁻¹; ¹H-NMR (60 MHz, CDCl₃) [symbol] 1.27 (s, 27H, Si[OC(CH₃)₃]₃), 2.12 (s, 3H, SCH₃), 2.4 to 2.6 (d, 2H, CH₂S) and 4.2 to 4.64 (m, 1H, α-proton).

Phenylalanine-tri-tert-butoxysilyl ester. Oil; Yield 15.7 g, 76%; Rf_{A}=0.73; ESMS, m/z 411 [M+H]⁺; IR (thin film) 3300-3100 (br, NH₂), 1745 (s, C═O str, ester), 1265 (s, C—O), 1100 (s, Si—O) cm⁻¹; ¹H-NMR (60 MHz, CDCl₃) δ1.27 (s, 27H, Si[OC(CH₃)₃]₃), 3.0 to 3.4 (d, 2H, benzylic-CH₂), 4.25 to 4.5 (m, 1H, α-proton) and 7.25 (m, 5H, aromatics).

Proline-tri-tert-butoxysilyl ester. Oil; Yield 13.5 g, 75%; Rf_{A}=0.80; ESMS, m/z 361 [M+H]⁺; IR (thin film) 1745 (s, C═O str, ester), 1265 (s, C—O), 1110 (s, Si—O) cm⁻¹; ¹H-NMR (60 MHz, CDCl₃) [symbol] 1.26 (s, 27H, Si[OC(CH₃)₃]₃), 1.7 to 2.45 (m, 4H, CH₂CH₂), 3.35 to 3.75 (m, 2H, CH₂ and 4.2 to 4.5 (m, 1H, α-proton).

Serine-(Bzl)-tri-tert-butoxysilyl ester. Oil; Yield 14.4 g, 65%; Rf_{A}=0.75; ESMS, m/z 441 [M+H]⁺; IR (thin film) 3500-3320 (br, NH₂), 1750 (s, C═O str, ester), 1260, 1195 (s, C—O), 1110 (s, Si—O) cm⁻¹; ¹H-NMR (60 MHz, CDCl₃) [symbol] 1.27 (s, 27H, Si[OC(CH₃)₃]₃), 3.6 to 3.7 (d, 2H, CH₂O), 4.0 to 4.3 (m, 3H, benzylic-CH₂, α-proton) and 7.28 (s, 5H, aromatics).

Threonine-(Bzl)-tri-tert-butoxysilyl ester. Oil; Yield 16.6 g, 73%; Rf_{A}=0.77; ESMS, 455 [M+H]⁺; IR (thin film) 3450-3320 (br, NH₂), 1750 (s, C═O str, ester), 1250, 1195 (s, C—O), 1110 (s, Si—O) cm⁻¹; ¹H-NMR (60 MHz, CDCl₃) [symbol] 1.2 to 1.8 (m, 30H, Si[OC (CH₃)₃]₃, CH₃), 4.0 to 4.55 (m, 3H, benzylic-CH₂, α-proton) and 7.29 (br.s, 5H).

Tryptophan-(formyl)-tri-tert-butoxysilyl ester. Oil; Yield 13.1 g, 51%; Rf_{A}=0.81; ESMS, m/z 479 [M+H]⁺; IR (thin film) 3500-3400 (br, NH₂), 1760, 1680 (s, C═O str, ester, formyl), 1200 (s, C—O), 1100 (s, Si-0) cm⁻¹.

Tryptophan-tri-tert-butoxysilyl ester. Oil; Yield 16.2 g, 72%; Rf_{A}=0.58; ESMS, m/z 452 [M+H]⁺; IR (thin film) 3500-3300 (br, NH₂), 1750 (s, C═O str, ester), 1200 (s, C—O), 1110 (s, Si—O) cm⁻¹.

Tyrosine-(Bzl)-tri-tert-butoxysilyl ester. Oil; Yield 20.2 g, 78%; Rf_{A}=0.90; ESMS, m/z 518 [M+H]⁺; IR (Thin film) 1750 (s, C═O str, ester), 1260, 1195 (s, C—O), 1110 (s, Si—O) cm⁻¹.

Valine-tri-tert-butoxysilyl ester. Oil; Yield 14.1 g, 78%; Rf_{A}=0.62; ESMS, m/z 363 [M+H]⁺; IR (thin film) 3500-3320 (br, NH₂), 1750 (s, C═O str, ester), 1250, 1195 (s, C—O), 1110 (s, Si—O) cm⁻¹; ¹H-NMR (60 MHz, CDCl₃) [symbol] 1.05 to 1.3 (m, 6H, C [CH₃]₂), 1.31 (s, 27H, Si[OC(CH₃)₃]₃), 2.15 to 2.40 (m, 1H, CH) and 3.85 to 4.10 (m, 1H, α-proton).

### Preparation of Resin

Derivatisation of tri-tert-butoxysilylamino acid ester with Merrifield chloromethyl resin was achieved in accordance with the methods described by Merrifield and others (Letsinger, R. L. and Kornet, M. J. (1963) J. Amer. Chem. Soc., 85, 3045-3046; Letsinger, R. L., Kornet, M. J., Mahadevan, V. and Jerina, D. M. (1964) J. Amer. Chem. Soc., 86, 5163; Felix, A. M. and Merrifield, R. B. (1970) J. Amer. Chem. Soc., 92, 1385-1391).

Chloromethylated co-polystyrene-2% divinylbenzene (5 g, 1 mmol/g) was suspended in 2-methoxyethanol (40 ml) and was treated with potassium acetate (1.4 g, 14.2 mmol) at 130° C. for 72 hours. The reaction mixture was allowed to cool to room temperature and filtered. The resin was washed with water (100 ml), methanol (100 ml), diethyl ether (100 ml), respectively, and dried (Infrared spectra, 1740 cm⁻¹). It was treated with NaOH (0.5M, 40 ml) at room temperature for 72 hours to complete the reaction. The resin was then washed with water, methanol, diethyl ether as before and dried under vacuo. The Infrared spectrum showed the absence of 1740 cm⁻¹ band. The hydroxymethyl resin was then treated with phosgene (20% solution in toluene, 40 ml, 80 mmol) at room temperature for 4 hours. The resin was filtered, washed thoroughly with diethyl ether and dried (Infrared, 1785 cm⁻¹).

### Attachment of the First Amino Acid to the Solid Support

Attachment of the first amino acid via its amino function was successfully achieved through the benzyloxycarbonyl linkage to Merrifield resin, by the method described by Felix and Merrifield ((1970) J. Amer. Chem. Soc., 92, 1385-1391) thus giving a peptide resin linkage that is stable enough to all reagents used during peptide synthesis, and is cleavable by the treatment of strong acid such as HF, HBr or TFMSA.

The resin substitutions were estimated by HBr cleavage (described below), and by the back-titration method after removal of the TBos ester (table 1). The general increase in substitution levels obtained for this work as compared to Jones was attributed to a higher substitution of the hydroxy moiety on the hydroxy methyl Merrifield resin.

### Estimation of the Level of Substitution of Amino Acid to the Resin by HBr Cleavage

After removal of the TBos ester, the resin was thoroughly washed with DCM (3×10 ml), Et₂O (2×10 ml) and dried under vacuum. Approximately 5 mg of resin was accurately weighed and placed in a clean micro-centrifuge tube. HBr in glacial acetic acid (0.2 ml, 30% wt/v) was added, the tube sealed and agitated by rotating mixer (10 rpm) for 4 hr at room temperature. The tube was carefully pierced in the cap and evacuated to dryness. The residue was diluted with MeOH (1 ml), filtered to remove the resin, washed with more MeOH (1 ml), and the filtrates pooled. An aliquot of this solution (50 μl) was subjected to a quantitative ninhydrin assay.

TABLE 1
The estimated substitution of the benzyloxycarbonyl
TBos ester resins.

| Benzyloxycarbonyl TBos | Estimated Substitution |
|---|---|
| ester resins | (mmol/g) |
| Alanine | 0.47 |
| Asparagine | 0.40 |
| Arginine (NO₂) | 0.45^{†} |
| Cysteine (MeOBzl) | 0.46 |
| Glycine | 0.35 |
| Isoleucine | 0.50 |
| Leucine | 0.50 |
| Lysine (NαFmoc) | 0.44 |
| Lysine (Z) | 0.30^{‡} |
| Methionine | 0.45 |
| Phenylalanine | 0.32 |
| Proline | 0.31 |
| Threonine (Bzl) | 0.35 |
| Tyrosine (Bzl) | 0.18 |
| Valine | 0.41 |

Peptide Synthesis

Having ascertained the optimal reaction conditions for this form of peptide synthesis, attention was focussed upon obtaining longer peptides. These were a 5-mer Leucine enkephalin; a 9-mer, Bradykinin; a 10-mer, the C-terminal of bovine rhodopsin; an 11-mer, a derivative of the active sequence of BPI (bactericidal/permeating increasing protein); and a 14-mer, the active portion of melattin. The peptides were chosen in part because when considered together, they contain nearly all the naturally occurring amino acids (except Asp, H is or Met). Furthermore, because some of the peptides are already well characterised by biological activity assays and by synthesis by conventional means by the inventors, and therefore serve as a direct comparison. To verify that the syntheses (monitored by back-titration unless otherwise stated) were proceeding according to their schedules, at intervals, some of the peptidyl-resin was cleaved by high HF or HBr and the product analysed by RP-HPLC, and mass spectrometry techniques.

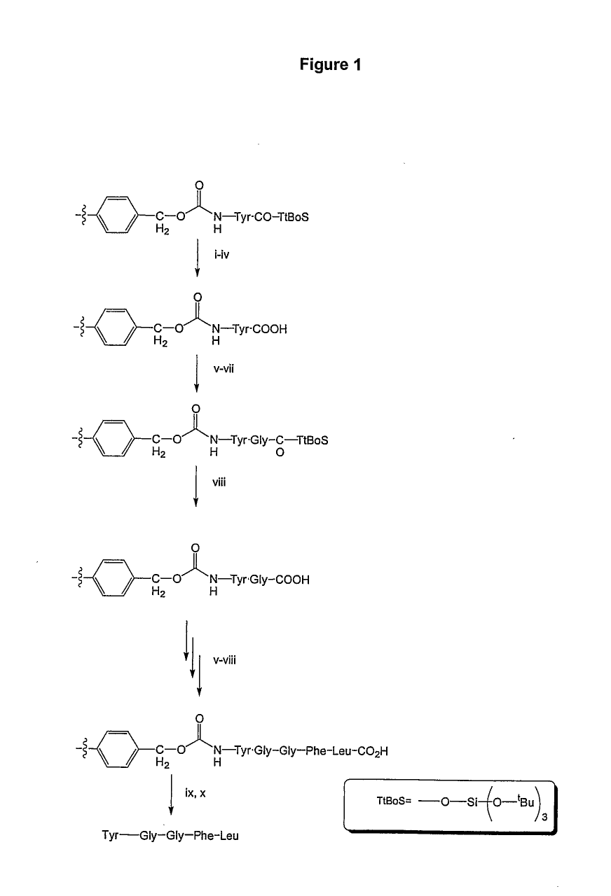

All peptides were assembled manually on the solid phase from the N to C direction (a reaction protocol for N to C synthesis is illustrated in FIG. 1 upon a 0.1 mmol scale).

FIG. 1 shows the synthesis of leucine-enkephalin on the solid phase in the N→C direction. The solvent was 6 mL throughout for 0.5 g of resin; reagents and conditions: I, wash, CH₂Cl₂ (2×1 min); ii, deprotect, 25% TFA-CH₂Cl₂ (2×5 min); iii, wash, CH₂Cl₂ (3×1 min), DMF (1 min); iv (optional), monitoring, remove 3-5 mg resin for assay; v, coupling, T-t-Bos amino acid (4 fold excess): BOP: HOBt:DIPEA (1:1:1:2 equiv), DMF, 60 min; vi, wash, DMF (2×1 min); vii, repeat iv; viii, repeat ii and iii; ix, cleavage, HF or TFMSA; x, purification, RP-HPLC. When necessary the amino acid derivatives were recoupled. Coupling and recoupling times were never longer than one hour.

The method involved washing, coupling and deprotection steps similar to that of conventional Boc solid phase peptide synthesis. The TBos group was used for temporary carboxyl protection of amino acids and side chain protecting groups were: Tyr (Bzl), Thr (Bzl), Lys (Z), Glu (OBzl), Ser (Bzl), Cys (MeOBzl), Trp (Formyl), and Arg (NO₂). All couplings were performed in DCM and whenever necessary, a second coupling was carried out. The peptides were cleaved by high HF, and purified by standard RP-HPLC. Following a brief description of the synthesis, related analytical data for the peptides could be found in table 2.

### Synthesis of Leu-Enkephalin

Leu-Enkephalin is an endogenous neurotransmitter with the sequence YGGFL. The peptide was synthesised on a tyrosine (Bzl)-derivatised resin (0.18 mmol/g) in 60% yield. The synthesis was examined after each coupling cycle as shown in Table 2. For comparison, the peptide was also synthesised by Fmoc chemistry (0.1 mmol scale, 65% yield).

TABLE 2
Stepwise synthesis (see also FIG. 1)

| | ESMS | | RP-HPLC | Percentage |
|---|---|---|---|---|
| Peptide Sequence | [M + H]⁺ | Yield (%) | RT (min) | coupling ^{a} |
| Tyr-Gly | | | 11.85 | 98 |
| Tyr-Gly-Gly | | | 10.01 | 100 |
| Tyr-Gly-Gly-Phe | | | 13.26 | 100 |
| Tyr-Gly-Gly-Phe-Leu | 556 | 60 | 14.98 | 98 |

^{a} As determined by back titration method (see below).

### Back Titration

Carboxyl groups in solution are quantified by a “back-titration” method (Skoog, D. A., West, D. M. and Holler, F. J., (1988) Fundamentals of Analytical Chemistry, 5^{th} edition, W. B. Saunders Company New York). The unknown carboxyl is treated with an excess of a known standard base solution, and the resultant mixture is titrated against a standard acid solution to neutrality. The amount of carboxyl originally present is then be calculated. To discover whether this method was applicable to the solid phase and TBos esters, a pilot study was performed which utilised an orthogonally protected Lysine TBos derivative, Nα-Fmoc-Lys-TBos (TBos esters being stable to piperidine). Once attached to a resin, the α-carboxyl or the α-amine could be specifically deprotected and assayed by either ninhydrin, or back-titration.

### Comparison and Estimation of Resin Substitution Via Back Titration, and Quantitative Ninhydrin Methods

The stability of the TBos esters to the standard reagents (0.01 M NaOH, and 0.001M HCl) for the time period for a titration were elucidated prior to testing on the solid phase by exposure and subsequent TLC examination at various time points.

Freshly prepared methyl chloroformate resin (substitution unknown, 200 mg) was reacted with N□-Fmoc-Lys-TBos, in the normal manner, joining the 6 amine to the resin. After extensive washing with DCM and drying under vacuum, approximately 5 mg of the functionalised resin was accurately weighed and underwent the back titration. [A qualitative ninhydrin assay upon approximately 5 mg of the resin gave a negative result]. The rest of the resin was split into two equal portions; one portion underwent Fmoc deprotection, the other was TBos deprotected. Both portions were extensively washed with DCM and dried under vacuum. Approximately 5 mg of each treated resin was accurately weighed. The Fmoc portion underwent a quantitative ninhydrin assay, the TBos deprotected portion underwent the back titration.

Ninhydrin: 1.8 mg resin, OD₅₇₀ (10× dilution)=0.521 corresponding to a substitution of 0.41 mmol/g.

Titration (protected resin): 3.8 mg resin took 9.8 mL 1 mmol HCl to neutralise in the back titration, corresponding to a value of 0.05 mmol/g background.

Titration (deprotected resin): 2.5 mg resin, took 8.9 mL 1 mM HCl to neutralise in the back titration, corresponding to a substitution of 0.44 mmol/g.

The result shown above was found to be highly reproducible, and demonstrated that the back-titration method is effective for the deprotected state of the resin (ie treatment with TFA followed by extensive washing with DCM) which leaves a protonated carboxyl on the resin.

The next step was to elucidate whether the back-titration could be performed upon a resin that has undergone a coupling reaction, or a partial reaction, and therefore be used to predict the extent of the reaction. The important factor to take into account for monitoring a coupling is that the unreacted carboxyl is likely to be in the activated state (the HOBt ester, when using BOP/HOBt as in this study), which is readily decomposed via base-hydrolysis, and in doing so, neutralises the base. In theory, the amount of base neutralised in this way should be quantifiable by the back-titration method, and the completeness of the coupling therefore calculated, having already ascertained the substitution of the resin.

An attempt at quantifying the extent of a coupling by performing back-titrations at various time points throughout a coupling reaction and verifying the result by cleavage, then RP-HPLC analysis of the products was only partially successful since it was difficult to control the extent of the reaction. However, employing the back-titration as an indicator whether a recoupling was required did result in a marked improvement to the yield and crude quality of the products obtained. In particular, the number of deletion peptides were reduced.

### Fragment Condensation Reaction on Resin

The following three examples illustrate aspects of the solid: solid technique.

### Comparative Example A

### Synthesis of NH₂-Lys-Thr-Glu-Thr-Ser-Gln-Val-Ala-Pro-Ala-OEt

Peptide KTETS was assembled on derivatised Merrifield resin from N to C direction as described above to give peptidyl resin, Resin-KTETS-COOH(XI).

Peptide H₂N-QVAPA-OEt was synthesised in a similar manner and cleaved from resin and purified by RP-HPLC. The latter fragment (XII) was then coupled to peptidyl resin (XI) in the standard manner to give, after cleavage and purification the 10 amino acid product H₂N-KTETSQVAPA-OEt (XIII).

### Example B

### Resin to Resin Coupling

The peptide KTET (XIV) was assembled on the derivatised Merrifield resin from N to C direction as above.

The peptide SQVAPA (XV) was assembled in C-N direction on WANG resin using Fmoc methodology.

The peptidyl resin (XIV) was added in slight excess to peptidyl resin (XV) in dimethylformamide (DMF) in the presence of coupling reagent BOP and base diisopropylethylamine (DIPEA) and was shaken for 90 minutes at room temperature. Solvents were removed by filtration and cleavage by HF gave the crude peptide, which was purified by RP HPLC and characterised by FIB Mass spectrometry to give peptide KTETSQVAPA (XVI) in excellent yield. This was further characterised by the synthesis of peptide (XVI) by Fmoc methodology, and co-injection with this material with the product of Example B. The two materials co-eluted.

### Example C

### Synthesis of Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg (XVII)

The peptidyl-resin—Gly-Ile-Gly-Ala-Val-Leu-Lys-Val-Leu-Thr-NH₂ (XVIII) was synthesised C to N by Fmoc methodology.

Thr-Gly-Leu-Pro-Ala-Leu-Ile-Ser-Trp-Ile-Lys-Arg-Lys-Arg-Resin (XIX) was synthesised using the N to C method and the two peptidyl resins were coupled as described above. The HF cleavage and purification gave peptide Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg (XVII).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A process for the preparation of a solid support-bound peptide of formula (I) 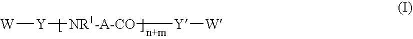
wherein
n is a positive integer
m is a positive integer
W and W′ are solid supports
Y and Y′ are linker groups
R¹ is hydrogen or a substituent, and may be the same or different,
and for each A, which may be the same or different,
i) A represents the amino acid residue; or
ii) A, taken together with R¹ and N, forms a heterocycle
comprising reacting a solid support-bound activated peptide of formula (II) with a solid support-bound peptide of formula (III); 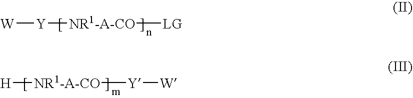
wherein LG is a leaving group.

2. A process according to claim 1 comprising a further step of cleaving the peptide (I) from the linker Y′ to give a solid support-bound peptide of formula (V) 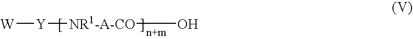
or a salt form thereof.

3. A process according to claim 1 comprising a further step of cleaving the peptide (I) from the linker Y to give a solid support-bound peptide of formula (IX) 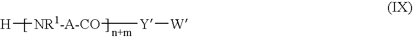
or a salt form thereof.

4. A process according to claim 1 comprising a further step of cleaving the peptide (1) from the linkers Y and Y′ to give a free peptide of formula (X) 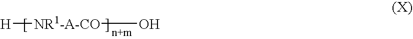
or a salt form thereof.

5. A process for the preparation of a compound of formula (VI) 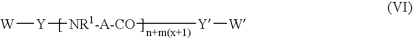
wherein W, Y, W′, Y′, R¹, A, n and m are as defined in claim 1 and x is a positive integer;
comprising the steps of
(a) reacting a solid support-bound activated peptide of formula (II) with a solid support-bound peptide of formula (III); 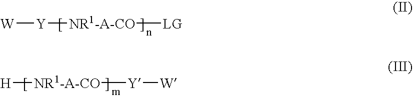
to give a solid support-bound peptide of formula (I) 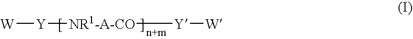
(b) cleaving the peptide (I) from the support W′ to give a solid support-bound peptide of formula (VII) 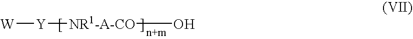
(c) treating solid support-bound peptide of formula (VII) with an activating agent to give a solid support-bound activated peptide of formula (VIII) 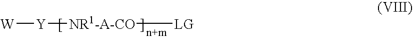
wherein LG is as defined in claim 1
(d) repeating steps (a), (b) and (c) x times.

6. A process for the preparation of a compound of formula (VI) 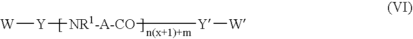
wherein W, Y, W′, Y′, R¹, A, n and m are as defined in claim 1 and x is a positive integer;
comprising the steps of
(a) reacting a solid support-bound activated peptide of formula (II) with a solid support-bound peptide of formula (III); 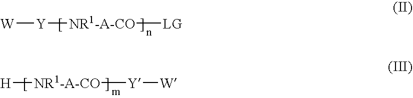
to give a solid support-bound peptide of formula (I) 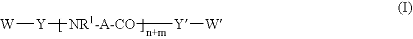
(b) cleaving the peptide (1) from the support W to give a solid support-bound peptide of formula (IX) 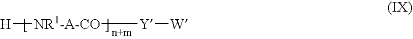
(c) repeating steps (a) and (b) x times.

7. A solid support-bound peptide of formula (I) as defined in claim 1.

8. A process for preparing a peptide or protein by solid phase synthesis comprising combining a sequence including one or more amino acids obtainable by C-N synthesis linked to a first resin, with an amino acid sequence including one or more amino acids obtainable by N-C synthesis linked to a second resin so as to create a native peptide link between unprotected N and unprotected C terminals of said amino acid sequences, and optionally releasing the resulting peptide from one or more of the linked resins so as to combine with further N-C or C-N sequences or to release the desired peptide or protein sequence.

9. A process according to claim 1 wherein the solid supports W and W′ are different.

10. (canceled)

11. (canceled)
